(19) United States
(12) Patent Application Publication
(10) Pub. No.: US 2025/0302537 A1
(43) Pub. Date: Oct. 2, 2025

(54) FEATURE AND ANALYSIS BASED DESIGN SCHEME FOR AN ORTHOPEDIC CUTTING INSTRUMENT

(71) Applicant: Zimmer, Inc., Warsaw, IN (US)

(72) Inventors: Robert E. Montgomery, South Bend, IN (US); Maged Awadalla, Warsaw, IN (US)

(21) Appl. No.: 19/087,104

(22) Filed: Mar. 21, 2025

Related U.S. Application Data
(60) Provisional application No. 63/571,662, filed on Mar. 29, 2024.

(51) Int. Cl. A61B 34/10; A61B 17/00; A61B 17/16

(52) U.S. Cl. CPC A61B 34/10; A61B 17/1615; A61B 2017/00526; A61B 2017/00725; A61B 2034/102

(57) ABSTRACT

Illustrated and discussed examples include a method of designing a cutting instrument for removing a portion of a bone of a patient. The method can include: receiving with a computing device, bone data; receiving with the computing device, operation data regarding one or more operating parameters for the cutting instrument; receiving a first plurality of device design parameters for the cutting instrument; performing a first analysis using the first plurality of device design parameters for the cutting instrument, the bone data and the operation data; altering one or more of the first plurality of device design parameters to a second plurality of device design parameters for the cutting instrument; performing a second analysis using the second plurality of device design parameters, the bone data and the operation data; and outputting a design of the cutting instrument after performing at least the first analysis and the second analysis.

### CLAIM OF PRIORITY

This application claims the benefit of U.S. Provisional Patent Application Ser. No. 63/571,662, filed on Mar. 29, 2024, the benefit of priority of which is claimed hereby, and which is incorporated by reference herein in its entirety.

### FIELD OF THE DISCLOSURE

The present disclosure relates generally to bone cutting instruments for use in joint replacements and the methods of design thereof. More specifically, the present disclosure relates to design methods and systems for such surgical instruments using simulating and modeling techniques as further discussed herein.

### BACKGROUND

The shoulder (e.g., glenohumeral) joint is the most mobile joint in the human body. In a healthy shoulder joint, the humeral head of the humerus articulates within the glenoid cavity of the scapula, which, together various soft tissues, allows the shoulder joint to articulate through a wide range of motion. However, through injury or disease, degradation of humeral or glenoid bone, or various soft tissues, often leads to corrective surgery to help restore joint functionality, such as in the form of a total shoulder arthroplasty or a reverse shoulder arthroplasty. Both total and reverse shoulder replacement surgeries involve removal of bone using reamers and/or other cutting instruments and the implantation of a prosthetic shoulder joint that is matched with the bio-kinematics of a patient.

Various types of shoulder implant systems are known including conventional or reverse joint replacement, revision shoulder arthroplasty and a partial (hemi) shoulder arthroplasty. For example, prosthetic shoulder joints include a humeral implant adapted for fixation to the humerus and a glenoid implant adapted for fixation to glenoid bone within the glenoid cavity of the scapula. In a total shoulder arthroplasty, the humeral implant generally includes a stem for insertion into the humeral medullary canal and a prosthetic humeral head secured thereto for replacing the natural humeral head; and the glenoid implant generally includes a baseplate adapted for fixation to glenoid bone and a prosthetic cup secured thereto to provide an articular surface for the prosthetic humeral head. In a reverse shoulder arthroplasty, the baseplate of the glenoid implant includes a spherical component, often called a glenosphere, and the stem of the humeral implant includes the prosthetic cup to provide an articular surface for the spherical component.

### SUMMARY

The present disclosure relates to bone cutting instruments. These instruments can be specifically designed for orthopedic surgical applications such as shoulder arthroplasty. The cutting instruments and the disclosed design methodologies advance the state of the art in glenoid reamer design and in the design of other bone cutting instruments. Thus, although the present examples are discussed in reference to a glenoid reamer, the techniques, methods and systems are also applicable to other bone cutting instruments or reamers used for other joints and bones of the human body. Similarly, although the present disclosure describes modeling that utilizes finite element analysis, the present disclosure contemplates the models discussed herein can utilize other known modeling techniques such as any one or more of: musculoskeletal modeling, analytical methods, multibody dynamics, modal analysis, boundary element method, finite difference method, probabilistic method, system-level simulation and/or experimental testing.

In preparing a bone cavity to receive an implant, such as by rasping and reaming, a surgeon can transmit large forces to the bone. These high forces can lead to bone fracture, especially in cases of poor bone quality, thin cortical thickness, unfavorable bone shape, oversized implants, etc. The thickness, density, and shape of the cortical bone, the presence of cancellous bone, sclerotic bone and/or osteoporotic bone directly impact the risk of fracture when preparing the bone for the implant or impacting the implant into the prepared bone. A bone with a thick cortex can better accommodate an implant that fills the canal more and can withstand higher impaction forces. In contrast, a bone with a thinner cortex or the presence of other bone types may fracture under similar conditions. Additionally, the morphology of the bone, shape of cutting instruments and implant influences fracture risk.

Challenges with existing reamer designs include the management of bone chips, stress distribution on the bone and the reamer, and the optimization of the cutting edges of the reamer for efficient bone removal without compromising the integrity of the instrument or the bone. Traditional designs have had difficulty balancing these factors, often resulting in designs that either remove bone too aggressively, risking bone integrity, or too conservatively, requiring more effort and time from the surgeon. Traditional glenoid reamers have utilized designs that may not optimally balance the removal of bone with the preservation of bone integrity and the durability of the reamer. For instance, the use of generally straight blades can lead to increased torque requirements and potential for instrument failure and/or bone fracture.

The present invention is rooted in a feature and analysis-based design scheme that leverages both computational and empirical methodologies to refine the design features (sometimes called parameters or geometry parameters herein) of the cutting instrument. This design scheme is aimed at achieving a balance between the mechanical stresses and/or strain exerted on the bone during reaming and the physical stresses experienced by the reamer, thereby enhancing the efficiency of bone removal, reducing the risk of instrument failure, reducing the possibility of undesired bone breakage and/or minimizing the physical strain on the surgeon or robotic system (by examining reaction forces computationally and empirically). The disclosed systems, apparatuses and methods can incorporate machine learning model(s) and simulations, or artificial intelligence (AI) developed based on data collected computationally and empirically and used to aid in the design techniques disclosed. The design processes can improve cutting instrument parameters to reduce likelihood of post-operative complications among other benefits.

Patient bone shape and material properties, patient's kinematic and kinetic, operation data and instrument data can be input into the computational modelling processes, systems and techniques discussed herein. The modelling not only simulates loading conditions and bone-instrument interface but can also be optimized based on user variability parameters (surgeon) and design parameters like the number of cutting blades of the cutting instrument; the length of each of the cutting blades of the cutting instrument; the cutting angle of each of the cutting blades of the cutting instrument; the relief angle of a cutting edge of each of the cutting blades of the cutting instrument; the curvature of each of the cutting blades of the cutting instrument; the number of notches of each of the cutting blades of the cutting instrument; the depth of each of the notches of each of the cutting blades of the cutting instrument; the first cross-sectional thickness of each of the cutting blades taken at or adjacent a middle of each cutting blade; and/or the second cross-sectional thickness of each cutting blade taken at or adjacent a connection with a hub.

Based on large population analysis of bone density variability in the proximal humerus, the instrument design (one or multiple pre-designed of the shelf instruments based on patient's proximal humorous bone density) will better match the patient's bone strength. The bone density will be collected from the patient's CT scan prior to the surgery. The bone density will determine which off the shelf cutter (a selection of size and shapes) to use based on prior knowledge gained from computational and empirical analysis that pairs bone density to optimal instrument design to use for this specific patient. Surgical outcomes including patient outcomes and recovery are thereby improved. Additional benefits recognized by the present disclosure include an ability to account for patient-specific factors like bone quality and biomechanics through machine learning and/or simulation/modeling, automated design optimization through machine learning, finite element analysis, and/or other modeling and a capability to identify and reinforce high-stress and/or strain regions with improved construction.

The full extent of the techniques discussed herein are described below with reference to the figures. The above is provided merely as a brief, non-limiting, summary of the disclosure.

### BRIEF DESCRIPTION OF THE FIGURES

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A is a first perspective view of a cutting instrument, a glenoid reamer, in accordance with at least one example of this disclosure.

FIG. 1B is a second perspective view of the cutting instrument of FIG. 1A.

FIG. 2 is a table illustrating various example geometry parameters of the cutting instrument of FIGS. 1A-1B that are subject to possible change, in accordance with at least one example of this disclosure.

FIG. 3 is a table illustrating various example options and/or considerations for modeling a bone of a patient, in accordance with at least one example of this disclosure.

FIG. 4 illustrates an example method of designing a cutting instrument, in accordance with at least one example of this disclosure.

FIG. 5 is a table illustrating various example options and/or considerations for modeling the cutting instrument with changes to the geometry parameters of FIG. 2, in accordance with at least one example of this disclosure.

FIG. 6 is a flow chart of another example method of designing a cutting instrument, in accordance with at least one example of this disclosure.

FIG. 7 illustrates an example method of designing cutting instruments using a machine learning model, in accordance with at least one example of this disclosure.

FIG. 8 illustrates an example schematic of a system including a computing device, in accordance with at least one example of this disclosure.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

During a shoulder replacement surgery (e.g., a shoulder arthroplasty), implantation of the humeral implant into the humerus first involves a resection (a cutting) of an articular portion thereof (e.g., the humeral head) to remove diseased or damaged bone and expose the medullary canal. The medullary canal, or other portions of the humerus, can then be reamed or rasped to prepare the humerus to receive the stem of the humeral implant. Implantation of the glenoid implant first involves reaming or other cutting of a glenoid surface within the glenoid cavity to prepare the scapula to receive or otherwise engage the baseplate of the glenoid implant.

The present disclosure utilizes a blend of innovation, iterative modeling and testing, and is premised on an understanding of both the mechanical and biological factors at play. Traditional reamer designs have limitations as discussed previously above in the SUMMARY.

To help address these limitations, among others, the present disclosure can provide a design process usable to optimize various cutting instruments, such as, but not limited to, glenoid reamers, to improve such cutting instruments. More particularly, the present disclosure discusses feature and analysis-based development scheme, a novel approach that seeks to reimagine an orthopedic instrument's development process. The design methodology leverages a combination of computational analysis, specifically design and analysis of computer experiment utilizing a multifactorial finite element analysis (FEA). This analysis can be done in combination with with Monte Carlo examination of design tolerance and computational analysis of other external factors (patient factors, surgeon factors, and manufacturing factors), and empirical bench testing to iteratively refine the design of the cutting instrument. The goal is to create a cutting instrument that not only minimizes stress on the glenoid bone but also reduces the torque required by the surgeon or robotic system, thereby enhancing the efficiency of bone removal and reducing the risk of instrument and/or bone failure.

The above discussion is intended to provide an overview of subject matter of the present patent application. It is not intended to provide an exclusive or exhaustive explanation of the invention. The description below is included to provide further information about the present patent application.

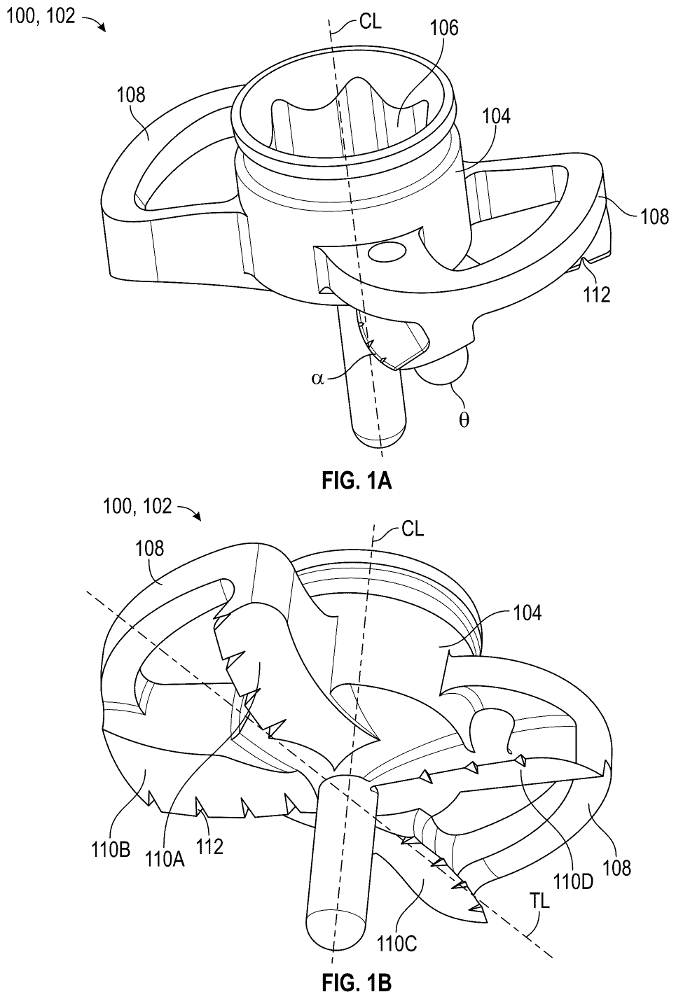

FIGS. 1A and 1B illustrate an example of a cutting instrument 100, a glenoid reamer 102, whose reaming design can be optimized for the bone using the methods, techniques and systems discussed further herein. The glenoid reamer 102 can include a hub 104, a coupling feature 106 (FIG. 1A), webs 108, a plurality of bone cutting blades 110A, 110B, 110C and 110D (labeled in FIG. 1B) and a centerline axis CL (axis of rotation). Each of the plurality of bone cutting blades 110A, 110B, 110C and 110D include notches 112, a curvature (discussed further below) and an angulation (such as rake/cutting angle and relief angle as further discussed below).

The hub 104 can form the coupling feature 106 as known in the art that can couple with a shaft or other mechanical feature. The hub 104 can form a main body or base for the glenoid reamer 102. The centerline axis CL can form an axis of rotation and can be aligned along a center of the hub 104. The plurality of bone cutting blades 110A, 110B, 110C and 110D can be coupled to and can extend outward from the hub 104. The webs 108 can be coupled to and can extend generally outward from the hub 104. The webs 108 can additionally couple to and can reinforce pairs of the respective of the plurality of bone cutting blades 110A, 110B, 110C and 110D. The webs 108 and the hub 104 can provide the glenoid reamer 102 with a general bowtie shape, for example, when viewed in plane from above.

As best illustrated in FIG. 1B, the plurality of bone cutting blades 110A, 110B, 110C and 110D can have several unique features. First, each of the plurality of bone cutting blades 110A, 110B, 110C and 110D has a curvature along an outer cutting edge thereof rather than the outer cutting edge simply extending radially straight outward from the hub 104. This curvature is illustrated by a tangent line TL taken along the outer cutting edge at or adjacent connection with the hub 104 for the bone cutting blade 110A. Thus, the bone cutting blade 110A at an outer end thereof is positioned further away from the tangent line TL than at or adjacent the connection with the hub 104. Put another way, the outer cutting edge extends in a curved line radially, like a fan or propeller blade. Second, the outer cutting edge of each of the plurality of bone cutting blades 110A, 110B, 110C and 110D at and adjacent to the connection with the hub 104 can be positioned to align/intersect with the centerline axis CL (axis of rotation). The TL can also be offset from this CL. This configuration is illustrated in FIG. 1B as the tangent line TL for the bone cutting blade 110A intersects with the centerline axis CL.

Additionally, the plurality of bone cutting blades 110A, 110B, 110C and 110D include the notches 112 that interrupt the outer cutting edge. The number, size and spacing of the notches 112 shown in FIGS. 1A and 1B is purely exemplary. The notches 112 can be V-shaped interruptions that encourage smaller bone chips to be released during reaming. The angulation including a relief angle is illustrated as angle θ along a face of the bone cutting blade 110A. A rake/cutting angle is also shown as angle α in FIG. 1B.

With these geometric features/parameters in mind, the methodologies, systems and techniques discussed herein can modify various of these geometric features/parameters using a combination of multifactorial computational analysis coupled with Monte Carlo examination of design tolerance and other external factors (patients, surgeons, and manufacturing) and empirical bench testing to iteratively refine the design of the cutting instrument to minimize stress on the glenoid bone and reduce the torque required by the surgeon or robotic system, thereby enhancing the efficiency of bone removal and reducing the risk of instrument and/or bone failure.

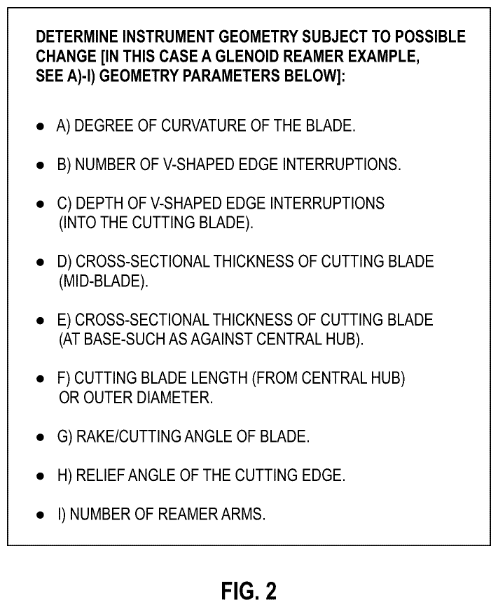

FIG. 2 presents a table of some of the geometric features/parameters that can be possibly altered during the design of the cutting instrument. It should be noted that changes in one of the features/parameters can affect others of the features/parameters such that simply changing a single or even two feature/parameter(s) is not contemplated for fully optimizing the configuration of the cutting instrument.

For example, providing additional notches (such as V-shaped edge interruptions) along the length of each cutting blade may improve function of reamer, as the more bone that can be cleared more quickly during reaming may improve bone cutting. However, too many notches acting as V-shaped edge interruptions may artificially make the reamer edge “dull” (as the more V-shaped edge interruptions are added, the less the “sharp” a length of the outer cutting edge becomes). In contrast, providing less notches could have the opposite affects discussed above.

A shorter outer cutting edge can result in a weaker cutting edge during reaming, with a greater chance of cutting edge or overall reamer fracture (as more force will be applied to the outer cutting edge with constant reaming pressure if the edge surface area is reduced). Similarly, regarding the depth of the notches or cross-sectional thickness of the bone cutting blade, providing deeper notches or relatively less cross-sectional area can weaken the outer cutting edge (because of less material in cross-section) but may improve bone cutting regarding application of the notches (capable of releasing larger/more bone chips, which are in the way of reaming). Additionally, the longer the bone cutting blade is, the greater the bending loads applied on the bone cutting blade. Such bending loads can be significant and will be measured empirically and included in the computational analysis. Stresses (on both the bone and the outer cutting edge) can be predicted as further discussed herein and balanced by multifactorial FEA coupled with Monte Carlo examination of design tolerance and other external factors (patient factors, surgeon factors, and manufacturing factors). Bench testing can be used to confirm suggested design adjustments for the next iteration/design adjustment.

Regarding the curvature of the bone cutting blade, if the bone cutting blade is more aggressive in reducing curvature (i.e. straight-line radially outward from centerline), there will be much more stress on the outer cutting edge of the bone cutting blade and the bone being reamed. In contrast, if the bone cutting blade and the outer cutting edge is curved (the bone cutting blade having a pronounced curvature instead of extending straight from centerline axis), the cutting instrument will engage the bone more gradually over the outer cutting edge length (instead of the whole outer cutting edge length engaging at the same time as the straight design does), causing less stress in the outer cutting edge and the bone. However, excessive curvature of the bone cutting blade could result in less aggressive reaming than is desired.

Regarding rake/cutting angle of the bone cutting blade, the angle on the bone cutting blade can affect the aggressiveness of cutting, with a more vertical blade being more aggressive at cutting (more bone removed per pass), while a flatter/less-vertical blade would be less aggressive. A more vertical bone cutting blade may put more stress on the blade/bone, leading to greater likelihood the blade and/or bone may fail, while vice versa would be the likely result for a relatively flatter bone cutting blade having a less aggressive angulation.

As the number of bone cutting blades is increased, the strength of the cutting instrument is improved as spreading the pressure over more bone cutting blades reduces the stress on each individual bone cutting blade (assuming same cross section). However, the gaps between bone cutting blades decrease for releasing bone chips/fragments during reaming, which can make reaming more challenging (e.g., may give user the feel that the reamer is dull if bone chips cannot be easily extracted from the cutting area).

FIG. 2 illustrates a number of geometry parameters that include but are not limited to: the number of cutting blades of the cutting instrument; the length of each of the cutting blades of the cutting instrument; the cutting angle of each of the cutting blades of the cutting instrument; the relief angle of a cutting edge of each of the cutting blades of the cutting instrument; the degree of curvature of each of the cutting blades of the cutting instrument; the number of notches of each of the cutting blades of the cutting instrument; the depth of each of the notches of each of the cutting blades of the cutting instrument; the first cross-sectional thickness of each of the cutting blades taken at or adjacent a middle of each cutting blade; and/or the second cross-sectional thickness of each cutting blade taken at or adjacent a connection with the hub. However, other parameters such as the general alignment or offset of the outer cutting edge with/from the axis of rotation of the cutting instrument can additionally be altered/changed.

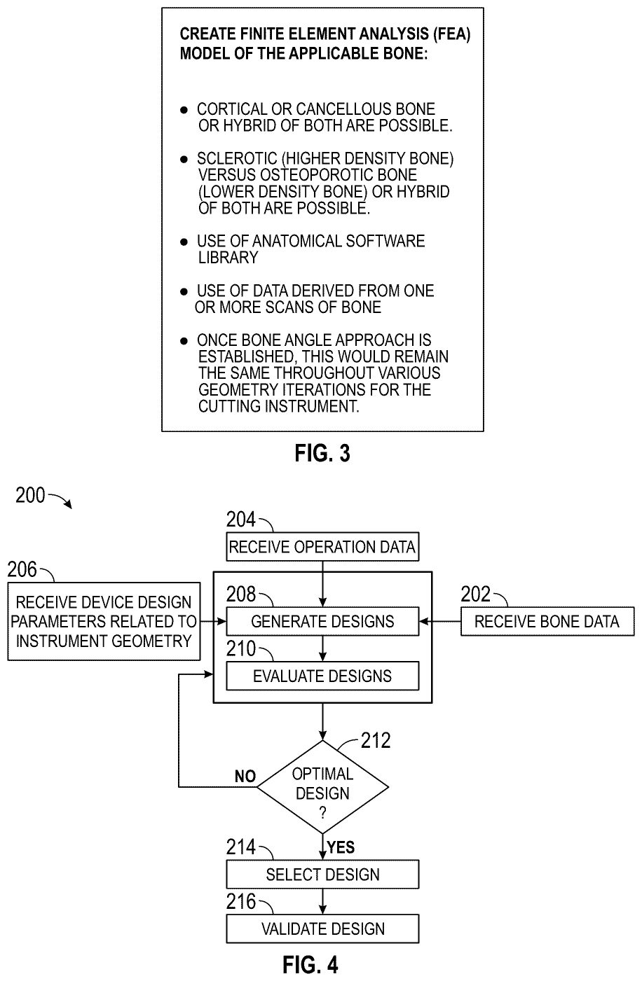

FIG. 3 is a table of various bone data that can be input into an FEA model or other model of the applicable bone. The bone data can be based upon one or more of: a combination of cortical and cancellous bone, presence or combination of sclerotic and osteoporotic bone, an angle of approach of the cutting instrument to the bone, for example. The bone data can be from an anatomic model. For example, the anatomic model can be an anatomic software library such as the ZiBRA™ Anatomical Modeling System. The ZiBRA™ Anatomical Modeling System can be used to analyze thousands of bones, both male and female, representing a diverse global population. Using ZiBRA a large population analysis of bone density variability in the proximal humorous can be conducted. Based on this analysis the large population can be divided into groups based on density and other characteristics. These groups will then be used in the instrument design (one or multiple pre-designed of the shelf instruments based on patient's proximal humorous bone characteristics). Prior to any surgery, the surgeon can request patient's CT scan prior which can be used to calculate bone density and other characteristics. The bone density will determine which off the shelf cutter (a selection of size and shapes) to use based on prior knowledge gained from computational and empirical analysis that pairs bone density to optimal instrument design to use for this specific patient. Additionally or alternatively, the bone data can be from one or more scans of the bone of the patient. For example, physical bone characteristics can be obtained from preoperative X-rays (imaging that produce images of the structures inside the body), CT scans (Computerized Tomography scans that use computers and rotating X-ray machines to create cross-sectional images of the body), MRIs (Magnetic Resonance Imaging) scans that use magnetic fields and radio waves to produce detailed images of the organs and tissues within the body) or other medical images. Bone data can include bone density (based on pixel values that may represent bone density) of one or more bones of the patient, various geometric parameters of one or more bones of the patient (e.g., the dimensions of features or surfaces thereof), or the relative anatomical locations of bone, ligament, or tendons within the patient, which can be determined or otherwise extracted. Bone data can optionally include additional patient information such as age, gender, osteoporosis, lifestyle characteristics (e.g., smoking or drinking habits), or other attributes that help predict fracture likelihood. Bone data can include bone angle of approach for the cutting instrument in engaging/contacting the bone. To reduce variables, once bone angle approach is established, this angle of approach would remain the same throughout various geometry iterations for the cutting instrument during the analysis and modeling process.

The methods discussed below can, in some examples, be generally representative of the design process as discussed with reference to any of the FIGURES (e.g., methods discussed previously or subsequently). The steps or operations of the methods are illustrated in a particular order for convenience and clarity; many of the discussed operations can be performed by multiple different actors, devices, or systems or can be done in parallel. It is understood that subsets of the operations discussed can be attributable to a single actor, device, or system and can be considered a separate standalone process or method.

FIG. 4 illustrates a method 200 of designing a cutting instrument for removing a portion of a bone of a patient. The method 200 can include receiving 202 with a computing device, bone data regarding a composition of the bone. Examples of this bone data were previously discussed with regard to FIG. 3. The method 200 can include receiving 204 with the computing device, operation data regarding one or more operating parameters for the cutting instrument. The operation data can include, but is not limited to, one or more of a maximum torque applied on the cutting instrument when removing the bone, a minimum torque applied on the cutting instrument when removing the bone, a ramping of torque of the cutting instrument when removing the bone, a speed of rotation of the cutting instrument when removing the bone and an oscillation rate of the cutting instrument when removing the bone. The method 200 can include receiving 206 a first plurality of device design parameters for the cutting instrument and performing a first analysis with the computing device using the first plurality of device design parameters for the cutting instrument, the bone data and the operation data. The performing the first analysis can be part of generating 208 a plurality of possible designs and evaluating the designs 210 as shown in FIG. 4. The method 200 can include altering one or more of the first plurality of device design parameters to a second plurality of device design parameters for the cutting instrument (part of the generating the plurality of possible designs of FIG. 4). The method 200 can include performing a second analysis with the computing device using the second plurality of device design parameters, the bone data and the operation data. The performing the second analysis can be part of generating 208 a plurality of possible designs and evaluating the designs 210 as shown in FIG. 4. Examples of the first plurality of device design parameters and the second plurality of device design parameters are discussed above with regard to FIGS. 1A-2.

The method 200 can include outputting by the computing device a design of the cutting instrument after performing at least the first analysis and the second analysis. The outputting by the computer device of the design can be the query step 212 and the selection step 214 of the method 200 of FIG. 4. If necessary or desired, the method 200 can optionally include altering one or more of the second plurality of device design parameters to a third plurality of device design parameters for the cutting instrument, performing a third analysis with the computing device using the third plurality of device design parameters, the operation data and the bone data and outputting by the computing device the design of the cutting instrument after performing at least the third analysis. The outputting by the computing device the design of the cutting instrument can include transmitting a file to a fabrication machine configured to manufacture the cutting instrument.

Optionally, the method 200 can include validation 216 of the design as further discussed herein. The validation 216 can include, for example, bench testing of a physical model and/or other optional additional analysis (examples further discussed herein). Thus, the validation 216 can include fabricating a physical model based upon the design of the cutting instrument and performing bench testing on the physical model using the bone data and the operation data to validate the design of the cutting instrument.

At least one of performing the first analysis and performing the second analysis can include simulating a loading of the cutting instrument and the bone by simulating one or more forces and moments on the cutting instrument and the bone with the computing device. The simulating the loading of the cutting instrument and the bone can include simulating a torsion load and a compression load to the cutting instrument and the bone. At least one of performing the first analysis and performing the second analysis can include determining with the computing device a first one or more portions of the cutting instrument and the bone that are subject to a maximum stress based upon the simulating the loading of the cutting instrument and the bone. At least one of performing the first analysis and performing the second analysis can include determining a deformation or strain value of a second one or more portions of the cutting instrument and the bone based upon the simulating the loading of the cutting instrument and the bone. As further discussed herein, optionally at least one of the first plurality of device design parameters and the second plurality of device design parameters are determined using a machine learning engine and wherein the computing device is configured to train the machine learning engine using related prior surgical procedures including at least one result or action taken by the computing device and at least one corresponding outcome.

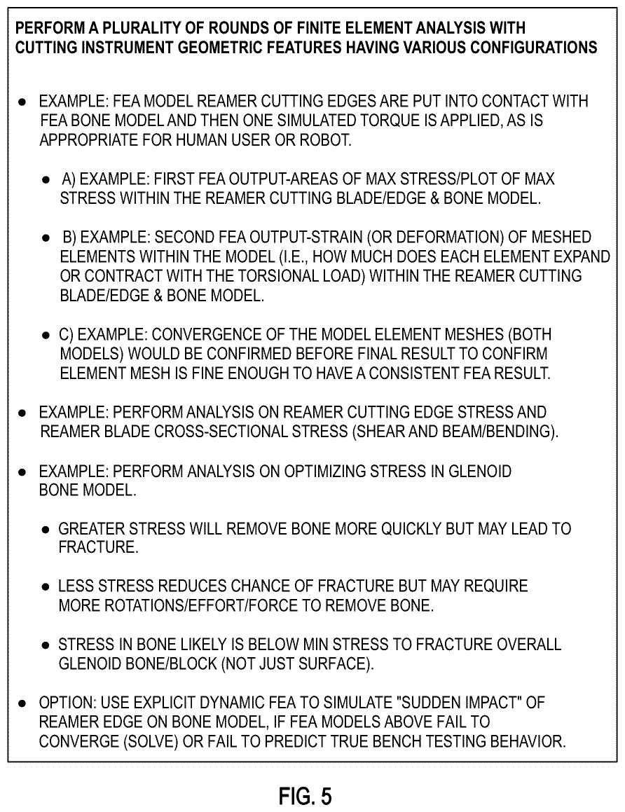

FIG. 5 is a table illustrating various example options and/or considerations for modeling the cutting instrument with changes to the geometry parameters of FIG. 2. For example, the modeling can include performing a plurality of rounds of FEA with cutting instrument geometric features having various configurations. The modeling can include FEA modeling with bone cutting edges put into contact with an FEA bone model and then simulate applied torque. The simulated torque can be appropriate for human user or robot. This loading can be simulated as a combination of compressive and shear load being placed on the cutting instrument according to one example. As an example, a first FEA output can be generated. This can include areas of max stress/plot of max stress within the reamer cutting blade/edge and the bone model. The model can generate a second FEA output including strain (or deformation) of meshed elements within the model. These meshed elements together can illustrate how much does each element expands or contracts with the torsional load within the reamer cutting blade/edge & bone model. Model validation and verification activity will be performed. This includes mesh sensitivity analysis to determine that the FEA outcomes are independent of mesh size. The modeling can include performing analysis on reamer cutting edge stress and reamer blade cross-sectional stress (shear and beam/bending). The multifactorial modeling analysis can additionally perform optimization on bone stress and strain, keeping in mind that greater stress will remove bone more quickly but can lead to bone fracture. Thus, use of less stress reduces the chance of fracture but can require relatively more effort to remove bone. The stress in bone is likely below min stress to fracture overall glenoid bone/block (not just surface). The modeling can optionally include using Explicit Dynamic FEA to simulate the dynamic behavior of the reamer edge on bone model, if FEA dynamic behavior and system energy is of interest to determine the probability of bone fracture.

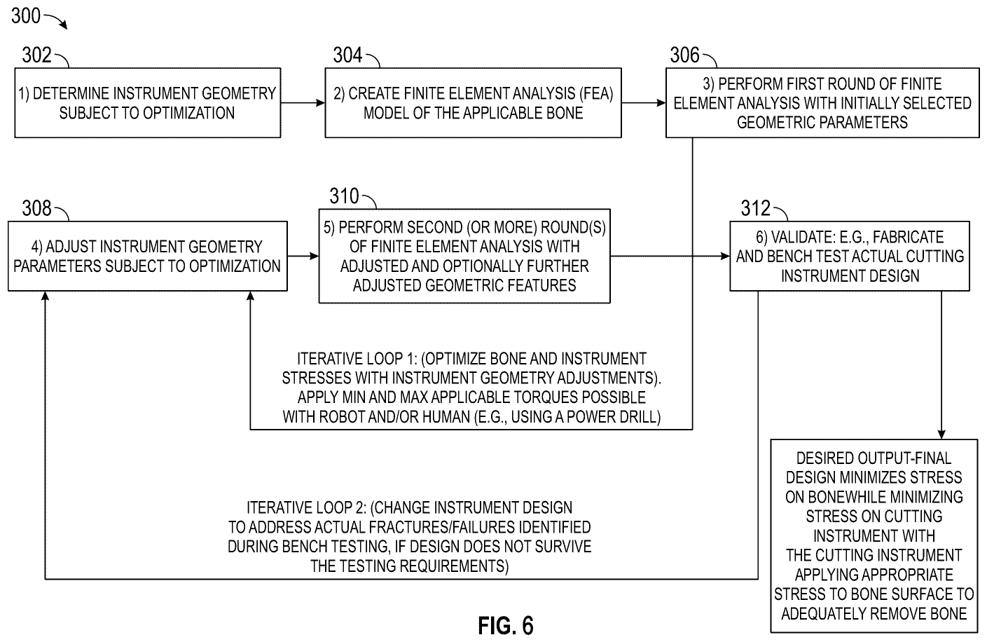

FIG. 6 illustrates a method 300 for design of a cutting instrument used for removing a portion of a bone of a patient. The method 300 can include determining 302 a cutting instrument geometry (e.g., shape, features, size, etc.) that are subject to design change. This geometry (e.g., a first geometry) can be provided to a computing device. The geometry can include one or more of: a) midline curvature of fan/propeller blade, b) number of v-shaped edge interruptions, c) depth of v-shaped edge interruptions into a cutting blade, d) cross-sectional thickness of the cutting blade at a mid-blade, e) cross-sectional thickness of the cutting blade at a base against a central housing, f) cutting blade length from the central housing or outer diameter, g) a rake/cutting angle of each of the cutting blades and h) a relief angle of a cutting edge.

The method 300 can include creating 304 a multifactorial FEA model of applicable bone and cutting instrument using the computing device. The method 300 can include performing 306 a first round of multifactorial FEA on the geometry (the initially selected geometric parameters) with the computing device to determine at least one or more areas of maximum stress on the geometry and the FEA model of the bone. The first round of FEA on the geometry can simulate minimum or maximum applicable applied torques with the intent of optimizing bone and instrument stresses with instrument geometry adjustments. The method 300 can include adjusting geometry of the cutting instrument based on at least the one or more areas of maximum stress (or other criteria). To this end, the method 300 provides for an iterative feedback loop (Iterative Loop 1 in FIG. 6) that adjusts/alters 308 the instrument geometry parameters subject to optimization.

The method 300 can perform 310 second (or more) round(s) of multifactorial FEA with adjusted parameters and optionally further adjusted geometric parameters. This can be driven by the outcomes of Monte Carlo analysis built on the multifactorial FEA model results. This can include performing a second round of finite element analysis on an adjusted geometry with the computing device to determine a strain (or deformation) of meshed elements of the geometry and the finite element analysis model of the bone and further adjusting the geometry of the cutting instrument based on at least the strain on the meshed elements. The method 300 can include validating 312 an output model simulating the cutting instrument with the computing device based upon the geometry as further adjusted based on at least the strain (or other criteria) on the meshed elements. This validation can include fabrication of a physical model and benching testing with the physical model. The method 300 provides another iterative feedback loop (Iterative Loop 2 in FIG. 6) that can change instrument design to address actual fractures/failures identified during bench testing, if the design does not survive the testing requirements of the bench testing. The method 300 can have a desired output where a final design of the cutting instrument minimizes stress on bone while minimizing stress on cutting instrument with the cutting instrument applying appropriate stress to bone surface to adequately remove bone.

To summarize, during multifactorial FEA coupled with Monte Carlo examination of design tolerance and analysis of other external factors (patient factors, surgeon factors, and manufacturing factors), various loads and/or moments may be placed on or applied to different iterations of the cutting instruments. Areas where stresses are above a predetermined level can be identified, and changed to lower or minimize the stresses. The process can be repeated with strain, deformation or another criteria. Modification can be made as desired based upon the various rounds of analysis.

Determining a final or optimal cutting instrument design as discussed above with regard to the method 200 and the method 300 (and the system discussed subsequently) can include generating one or more designs for the cutting instrument. Generating the plurality of designs can include passing, as inputs, various patient data, operation data and cutting instrument related parameters, such as determined or extracted from data into a machine learning (hereinafter “ML”) model (e.g., a machine learning artificial intelligence model), and receiving or otherwise obtaining suggested design(s) including particular cutting instrument parameters as outputs from the ML model. In some examples, the generating the one or more designs can include generating a plurality of different design options by generating a number of different cutting instrument designs, using the ML model. These plurality of designs can have different permutations of various cutting instrument parameters for particular types of bone, specific-patient characteristics or the like.

The methods 200 and 300 can include constructing the final or optimal cutting instrument. Constructing the cutting instrument can include exporting the final design parameters for a selected optimal design. For example, the design parameters of the cutting instrument can be exported or otherwise transmitted via computing device to an automated manufacturing device such as, but not limited to, a three-dimensional printer, a computer numerical controlled (CNC) milling machine, or other devices or systems. In some examples, the cutting instrument can be constructed using additive manufacturing, such as built layer by layer from titanium or cobalt-chrome. In some examples, fabrication can include post-processing, such as surface finishing or sterilization, to help ensure that the cutting instrument meets specifications and is safe, which may include fatigue testing or material characterization.

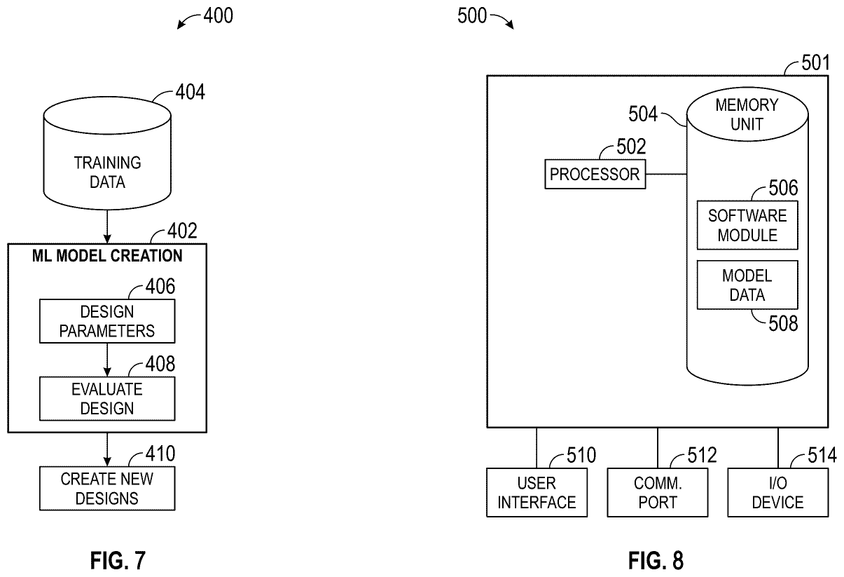

FIG. 7 illustrates an example method 400 for designing cutting instrument, in accordance with at least one example of this disclosure. The method 400 can, in some examples, be generally representative of the design processes, as discussed with reference to any of FIGS. 1A-6 above. The method 400 can include a first stage 402 and a second stage 410. The first stage 402 can include creation of an ML model. In some examples, the first stage 402 can include a first step 406 and a second step 408. In some examples, the first step 406 can include receiving or otherwise using inputs from a training data set 404. In some examples, the first step 406 can include defining cutting instrument design parameters and the second step 408 can include evaluating one or more implant designs under differing bone and/or operation criteria. The first step 406 and the second step 408 can be similar to, or can otherwise include, any of various aspects of, the first stage 402, the second stage 410, and a third stage (if used), respectively, discussed with respect to any of the methods discussed above. In other words, the ML model creation operation (the first stage 402) can utilize training data that can include any of the geometry parameters, bone data, operations data discussed above or data derivable from the bone data, operation data, geometry parameters or a combination thereof.

During the first stage 402, receiving or using inputs from the training data set 404 or other input data. Other input data can include input data related to or identifying the surgical procedure. As such, creation of the ML model can include defining or inputting the surgical procedure. Additionally, such data can include various of the other data discussed above. The first stage 402 can include generating a model. The model can allow the various inputs (e.g., the data discussed above including the cutting instrument geometry parameters and other data). The model can be used to output various cutting instrument designs. These designs are generated, such as during the second stage 410. For example, the various inputs can be entered into the ML model, and the ML model can, in return, output one or more possible cutting instrument designs. The various inputs can be part of a query to a database or repository. Using the various inputs, the ML model can return or output one or more cutting instrument designs that had the closest inputs to the various inputs utilized in the present surgery.

In some examples, the first stage 402 can include generating an initial guess for the parameters for the ML model based on the inputs. The parameters of the ML model can be optimized from the initial guess for the ML model and the plurality of input parameters. For example, for a given bone size, or a given bone quality, of the bone of the patient, an initial guess for a number, location, or other attributes of cutting instrument features, such as, among others, the number of cutting blades of the cutting instrument, the length of each of the cutting blades of the cutting instrument, the cutting angle of each of the cutting blades of the cutting instrument, the relief angle of a cutting edge of each of the cutting blades of the cutting instrument, the curvature of each of the cutting blades of the cutting instrument, the number of notches of each of the cutting blades of the cutting instrument, the depth of each of the notches of each of the cutting blades of the cutting instrument, the first cross-sectional thickness of each of the cutting blades taken at or adjacent a middle of each cutting blade and a second cross-sectional thickness of each cutting blade taken at or adjacent a connection with a hub, can be made. These parameters can then be subjected to FEA or other modeling to confirm the initial guess.

The training data 404 can be divided into various subsets of data for training and testing. A first subset (e.g., a training subset) of the training data 404 can be used to build various models of cutting instruments and/or bone models that have previously been tested using FEA. A second subset (e.g., a validation subset) of the training data 404 can then be used to validate the various models and/or any ML models that are created or generated in accordance with the present disclosure. In some examples, the training data 404 can include various of the different types of data discussed previously including operation and bone data. The bone data can include bone preservation data, anatomical data or other data obtained via CT, MRI, X-ray scans, or other imaging techniques. In one such example, one or more scans of one patient's anatomy can be received for a plurality of patients. The various input parameters can then be extracted from the one or more scans of the patient's anatomy for each of the plurality of patients, and subsequently saved as part of the training data 404. The ML model(s) can be exported for later use as needed.

The second stage 410 can include creating new cutting instrument design(s). In one example, the second stage 410 can include creating one or more generic designs. The generic designs can be based on, for example, average bone density and operation data such as average load applied on the bone of the patient. These generic design(s) can then be scaled to create a variety of different standardized sizes for a range of sizes that can be selected by a surgeon to fit a population of different patients. In other examples, the second stage 410 can include creating patient-specific designs based upon particular patient's bone data or different modified standard designs that specifically target particular segments of the population or are specifically configured for use with a robot v. human surgeon.

FIG. 8 illustrates an example schematic of a system 500 upon which the methods or techniques discussed can be applied. The system 500 can include a computing device 501, in accordance with at least one example of this disclosure. As shown in FIG. 8, the computing device 501 can include a processor 502 and a memory unit 504. The memory unit 504 can include a software module 506 and model data 508. While executing on the processor 502, the software module 506 can perform processes for generating one or more models, such as, but not limited to, those discussed above, performing FEA or other analysis on one or more generated designs. The model data 508 can include, but is not limited to, training data, such as training data 404 (FIG. 7), design parameters used as inputs, databases, models generated using various cutting instrument designs, or other data or information. The computing device 501 can also include a user interface 510, a communications port 512, and an input/output (I/O) device 514.

The user interface 510 can include any number of devices that allow a user to interface with the computing device 501. Some non-limiting examples of the user interface 510 can, but are not limited to, a keypad, a microphone, or a display (e.g., a touchscreen or otherwise). The communications port 512 can allow the computing device 501 to communicate with various information sources and devices, such as, but not limited to, remote computing devices, such as servers or other remote computers. For example, such remote computing devices can maintain data, such as model data, that can be retrieved by the computing device 501 using the communications port 512. Some non-limiting examples of the communications port 512 can include, but are not limited to, ethernet cards (e.g., wireless or wired), BLUETOOTH® transmitters and receivers, or near-field communications modules. The I/O device 514 can allow the computing device 501 to receive and output information. Some non-limiting examples of the I/O device 514 can include, a camera (e.g., still camera or a video camera), or fingerprint or other biometric scanners. For example, the I/O device 514 can allow the computing device 501 to directly receive patient data from a CT scanning device, an X-ray machine, or other imaging devices.

Thus, the system 500 can include at least one processor (processor 502) and at least one memory (memory unit 504). The memory can store instructions that, when executed by the at least one processor, cause the at least one processor to perform actions. These actions can include causing the processor to receive bone data regarding a composition of the bone, receive operation data regarding one or more operating parameters for the cutting instrument, receive a first plurality of device design parameters for the cutting instrument, perform a first analysis using the first plurality of device design parameters for the cutting instrument, the bone data and the operation data, alter one or more of the first plurality of device design parameters to a second plurality of device design parameters for the cutting instrument, perform a second analysis using the second plurality of device design parameters, the bone data and the operation data and output a design of the cutting instrument after performing at least the first analysis and the second analysis as discussed previously herein.

The instructions, when executed by the at least one processor, cause at least one processor to produce a file used to fabricate a physical model based upon the design of the cutting instrument. The instructions, that when executed by at least one processor, can cause the at least one processor to simulate a loading of the cutting instrument and the bone by simulating one or more forces and moments on the cutting instrument and the bone. The simulation of the loading of the cutting instrument and the bone includes simulating a torsion load and a compression load to the cutting instrument and the bone. The instructions that, when executed by at least one processor, can cause at least one processor to determine a first one or more portions of the cutting instrument and the bone that are subject to a maximum stress based upon simulation of the loading of the cutting instrument and the bone. The instructions that, when executed by the at least one processor, can cause at least one processor to determine a deformation or strain value of a second one or more portions of the cutting instrument and the bone based upon simulation of the loading of the cutting instrument and the bone. The instructions can cause at least one processor to alter one or more of the second plurality of device design parameters to a third plurality of device design parameters for the cutting instrument, perform a third analysis using the third plurality of device design parameters and the bone data and output the design of the cutting instrument after performing at least the third analysis. The output can include transmitting a file to a fabrication machine configured to manufacture the cutting instrument.

The foregoing systems, methods and devices are merely illustrative of the components, interconnections, communications, functions, etc. that can be employed in carrying out examples in accordance with this disclosure. Different types and combinations of electronics devices, computers including clients and servers, instruments, and other systems and devices can be employed in examples according to this disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided.

Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### EXAMPLES

The following, non-limiting examples, detail certain aspects of the present subject matter to solve the challenges and provide the benefits discussed herein, among others.

Example 1 is a method of designing a cutting instrument for removing a portion of a bone of a patient, optionally comprising: receiving with a computing device, bone data regarding a composition of the bone; receiving with the computing device, operation data regarding one or more operating parameters for the cutting instrument; receiving a first plurality of device design parameters for the cutting instrument; performing a first analysis with the computing device using the first plurality of device design parameters for the cutting instrument, the bone data and the operation data; altering one or more of the first plurality of device design parameters to a second plurality of device design parameters for the cutting instrument; performing a second analysis with the computing device using the second plurality of device design parameters, the bone data and the operation data; and outputting by the computing device a design of the cutting instrument after performing at least the first analysis and the second analysis.

In Example 2, the subject matter of Example 1 optionally includes, fabricating a physical model based upon the design of the cutting instrument; and performing bench testing on the physical model using the bone data and the operation data to validate the design of the cutting instrument.

In Example 3, the subject matter of Examples 1-2 optionally includes, wherein at least one of performing the first analysis and performing the second analysis includes simulating a loading of the cutting instrument and the bone by simulating one or more forces and moments on the cutting instrument and the bone with the computing device.

In Example 4, the subject matter of Example 3 optionally includes, wherein the simulating the loading of the cutting instrument and the bone includes simulating a torsion load, a compression load and a shear load to the cutting instrument and the bone.

In Example 5, the subject matter of Examples 3-4 optionally includes, wherein at least one of performing the first analysis and performing the second analysis includes determining with the computing device a first one or more portions of the cutting instrument and the bone that are subject to a maximum stress based upon the simulating the loading of the cutting instrument and the bone.

In Example 6, the subject matter of Examples 3-5 optionally includes, wherein the at least one of performing the first analysis and performing the second analysis includes determining a deformation or strain value of a second one or more portions of the cutting instrument and the bone based upon the simulating the loading of the cutting instrument and the bone.

In Example 7, the subject matter of Examples 1-6 optionally includes, wherein the bone data is derived from at least one of an anatomic library of data and a scan of the bone of the patient.

In Example 8, the subject matter of Examples 1-7 optionally includes, wherein the first plurality of device design parameters and the second plurality of device design parameters includes at least two or more of: a number of cutting blades of the cutting instrument; a length of each of the cutting blades of the cutting instrument; a cutting angle of each of the cutting blades of the cutting instrument; a relief angle of a cutting edge of each of the cutting blades of the cutting instrument; a curvature of each of the cutting blades of the cutting instrument; a number of notches of each of the cutting blades of the cutting instrument; a depth of each of the notches of each of the cutting blades of the cutting instrument; a first cross-sectional thickness of each of the cutting blades taken at or adjacent a middle of each cutting blade; and a second cross-sectional thickness of each cutting blade taken at or adjacent a connection with a hub.

In Example 9, the subject matter of Examples 1-8 optionally includes, altering one or more of the second plurality of device design parameters to a third plurality of device design parameters for the cutting instrument; performing a third analysis with the computing device using the third plurality of device design parameters, the operation data and the bone data; and outputting by the computing device the design of the cutting instrument after performing at least the third analysis.

In Example 10, the subject matter of Examples 1-9 optionally includes, wherein outputting by the computing device the design of the cutting instrument includes transmitting a file to a fabrication machine configured to manufacture the cutting instrument.

In Example 11, the subject matter of Examples 1-10 optionally includes, wherein the bone data is based upon one or more of: a combination of cortical and cancellous bone of a glenoid of the patient, a combination of sclerotic and osteoporotic bone of the glenoid of the patient and an angle of approach of the cutting instrument to the bone.

In Example 12, the subject matter of Examples 1-11 optionally includes, wherein the operation data includes one or more of a maximum torque applied on the cutting instrument when removing the bone, a minimum torque applied on the cutting instrument when removing the bone, a ramping of torque of the cutting instrument when removing the bone, a speed of rotation of the cutting instrument when removing the bone and an oscillation rate of the cutting instrument when removing the bone.

In Example 13, the subject matter of Examples 1-12 optionally includes, wherein at least one of the first plurality of device design parameters and the second plurality of device design parameters are determined using a machine learning engine and wherein the computing device is configured to train the machine learning engine using related prior surgical procedures including at least one result or action taken by the computing device and at least one corresponding outcome.

Example 14 is a system for designing a cutting instrument for removing a portion of a bone of a patient, optionally comprising: at least one processor; and at least one memory storing instructions that, when executed by the at least one processor, cause the at least one processor to perform actions comprising: receive bone data regarding a composition of the bone; receive operation data regarding one or more operating parameters for the cutting instrument; receive a first plurality of device design parameters for the cutting instrument; perform a first analysis using the first plurality of device design parameters for the cutting instrument, the bone data and the operation data; alter one or more of the first plurality of device design parameters to a second plurality of device design parameters for the cutting instrument; perform a second analysis using the second plurality of device design parameters, the bone data and the operation data; and output a design of the cutting instrument after performing at least the first analysis and the second analysis.

In Example 15, the subject matter of Example 14 optionally includes, wherein the instructions, when executed by the at least one processor, cause the at least one processor to produce a file used to fabricate a physical model based upon the design of the cutting instrument.

In Example 16, the subject matter of Examples 14-15 optionally includes, wherein at least one of the first analysis and the second analysis includes instructions that when executed by the at least one processor, cause the at least one processor to simulate a loading of the cutting instrument and the bone by simulating one or more forces and moments on the cutting instrument and the bone.

In Example 17, the subject matter of Example 16 optionally includes, wherein simulation of the loading of the cutting instrument and the bone includes simulating a torsion load, a compression load and a shear load to the cutting instrument and the bone.

In Example 18, the subject matter of Examples 16-17 optionally includes, wherein the instructions that when executed by the at least one processor, cause the at least one processor to determine a first one or more portions of the cutting instrument and the bone that are subject to a maximum stress based upon simulation of the loading of the cutting instrument and the bone.

In Example 19, the subject matter of Examples 16-18 optionally includes, wherein the instructions that when executed by the at least one processor, cause the at least one processor to determine a deformation or strain value of a second one or more portions of the cutting instrument and the bone based upon simulation of the loading of the cutting instrument and the bone.

In Example 20, the subject matter of Examples 14-19 optionally includes, wherein the bone data is derived from at least one of an anatomic library of data and a scan of the bone of the patient.

In Example 21, the subject matter of Examples 14-20 optionally includes, wherein the first plurality of device design parameters and the second plurality of device design parameters includes at least two or more of: a number of cutting blades of the cutting instrument; a length of each of the cutting blades of the cutting instrument; a cutting angle of each of the cutting blades of the cutting instrument; a relief angle of a cutting edge of each of the cutting blades of the cutting instrument; a curvature of each of the cutting blades of the cutting instrument; a number of notches of each of the cutting blades of the cutting instrument; a depth of each of the notches of each of the cutting blades of the cutting instrument; a first cross-sectional thickness of each of the cutting blades taken at or adjacent a middle of each cutting blade; and a second cross-sectional thickness of each cutting blade taken at or adjacent a connection with a hub.

In Example 22, the subject matter of Examples 14-21 optionally includes, wherein the instructions when executed by the at least one processor, cause the at least one processor to perform actions comprising: alter one or more of the second plurality of device design parameters to a third plurality of device design parameters for the cutting instrument; perform a third analysis using the third plurality of device design parameters, the operation data and the bone data; and output the design of the cutting instrument after performing at least the third analysis.

In Example 23, the subject matter of Examples 14-22 optionally includes, wherein the output includes transmitting a file to a fabrication machine configured to manufacture the cutting instrument.

In Example 24, the subject matter of Examples 14-23 optionally includes, wherein the bone data is based upon one or more of: a combination of cortical and cancellous bone of a glenoid of the patient, a combination of sclerotic and osteoporotic bone of the glenoid of the patient and an angle of approach of the cutting instrument to the bone.

In Example 25, the subject matter of Examples 14-24 optionally includes, wherein the operation data includes one or more of a maximum torque applied on the cutting instrument when removing the bone, a minimum torque applied on the cutting instrument when removing the bone, a ramping of torque of the cutting instrument when removing the bone, a speed of rotation of the cutting instrument when removing the bone and an oscillation rate of the cutting instrument when removing the bone.

In Example 26, the subject matter of Examples 14-25 optionally includes, wherein at least one of the first plurality of device design parameters and the second plurality of device design parameters are determined using a machine learning engine and wherein the at least one processor is configured to train the machine learning engine using related prior surgical procedures including at least one result or action taken by the at least one processor and at least one corresponding outcome.

Example 27 is a method for design of a cutting instrument used for removing a portion of a bone of a patient, optionally comprising: providing to a computing device a geometry of the cutting instrument, wherein the geometry comprises one or more of: a) midline curvature of fan/propeller blade, b) number of v-shaped edge interruptions, c) depth of v-shaped edge interruptions into a cutting blade, d) cross-sectional thickness of the cutting blade at a mid-blade, e) cross-sectional thickness of the cutting blade at a base against a central housing, f) cutting blade length from the central housing or outer diameter, g) a rake/cutting angle of each of the cutting blades and h) a relief angle of a cutting edge; creating a finite element analysis model of the bone using the computing device; performing a first round of finite element analysis on the geometry with the computing device to determine at least one or more areas of maximum stress on the geometry and the finite element analysis model of the bone; adjusting geometry of the cutting instrument based on at least the one or more areas of maximum stress; performing a second round of finite element analysis on an adjusted geometry with the computing device to determine a strain of meshed elements of the geometry and the finite element analysis model of the bone; further adjusting the geometry of the cutting instrument based on at least the strain on the meshed elements; and validating an output model simulating the cutting instrument with the computing device based upon the geometry as further adjusted based on at least the strain on the meshed elements.

In Example 28, the subject matter of Example 27 optionally includes, wherein validating the output model includes fabricating a physical model of the cutting instrument based upon the output model and performing bench testing using the physical model.

In Example 29, the subject matter of Examples 27-28 optionally includes, wherein the finite element analysis model of the bone is derived from at least one of an anatomic library of data and a scan of the bone of the patient.

In Example 30, the subject matter of Examples 27-29 optionally includes, wherein the finite element analysis model is a multifactorial finite element analysis.

In Example 31, the subject matter of Example 30 optionally includes, conducting a Monte Carlo examination of design tolerance and conducting further analysis of other external factors.

In Example 32, the subject matter of Examples 27-31 optionally includes, adjusting the geometry at least a third time; performing a third round of finite element analysis on an adjusted geometry with the computing device; and outputting by the computing device the design of the cutting instrument after performing at least the third round of finite element analysis.

In Example 33, the subject matter of Examples 27-32 optionally includes, transmitting a file to a fabrication machine configured to manufacture the cutting instrument.

In Example 34, the subject matter of Examples 27-33 optionally includes, wherein the finite element analysis model of the bone is based upon one or more of: a combination of cortical and cancellous bone of a glenoid of the patient, a combination of sclerotic and osteoporotic bone of the glenoid of the patient and an angle of approach of the cutting instrument to the bone.

In Example 35, the subject matter of Examples 27-34 optionally includes, applying operation data to the output model simulating the cutting instrument and the finite element analysis model of the bone, wherein the operation data includes one or more of a maximum torque applied on the cutting instrument when removing the bone, a minimum torque applied on the cutting instrument when removing the bone, a ramping of torque of the cutting instrument when removing the bone, a speed of rotation of the cutting instrument when removing the bone and an oscillation rate of the cutting instrument when removing the bone.

In Example 36, the subject matter of Examples 27-35 optionally includes, wherein at least one of the adjusting geometry of the cutting instrument and the further adjusting the geometry of the cutting instrument are determined using a machine learning engine and wherein the computing device is configured to train the machine learning engine using related prior surgical procedures including at least one result or action taken by the computing device and at least one corresponding outcome.

Example 37 is at least one machine-readable medium including instructions that, when executed by processing circuitry, cause the processing circuitry to perform operations to implement of any of Examples 1-36.

Example 38 is an apparatus comprising means to implement of any of Examples 1-36.

Example 39 is a system to implement of any of Examples 1-36.

Example 40 is a method to implement of any of Examples 1-36.

What is claimed is:

1. A method of designing a cutting instrument for removing a portion of a bone of a patient, comprising:
receiving with a computing device, bone data regarding a composition of the bone;
receiving with the computing device, operation data regarding one or more operating parameters for the cutting instrument;
receiving a first plurality of device design parameters for the cutting instrument;
performing a first analysis with the computing device using the first plurality of device design parameters for the cutting instrument, the bone data and the operation data;
altering one or more of the first plurality of device design parameters to a second plurality of device design parameters for the cutting instrument;
performing a second analysis with the computing device using the second plurality of device design parameters, the bone data and the operation data; and
outputting by the computing device a design of the cutting instrument after performing at least the first analysis and the second analysis.

2. The method of claim 1, further comprising:
fabricating a physical model based upon the design of the cutting instrument; and
performing bench testing on the physical model using the bone data and the operation data to validate the design of the cutting instrument.

3. The method of claim 1, wherein at least one of performing the first analysis and performing the second analysis includes simulating a loading of the cutting instrument and the bone by simulating one or more forces and moments on the cutting instrument and the bone with the computing device.

4. The method of claim 3, wherein the simulating the loading of the cutting instrument and the bone includes simulating a torsion load, a compression load and a shear load to the cutting instrument and the bone.

5. The method of claim 3, wherein at least one of performing the first analysis and performing the second analysis includes determining with the computing device a first one or more portions of the cutting instrument and the bone that are subject to a maximum stress based upon the simulating the loading of the cutting instrument and the bone.

6. The method of claim 3, wherein the at least one of performing the first analysis and performing the second analysis includes determining a deformation or strain value of a second one or more portions of the cutting instrument and the bone based upon the simulating the loading of the cutting instrument and the bone.

7. The method of claim 1, wherein the bone data is derived from at least one of an anatomic library of data and a scan of the bone of the patient.

8. The method of claim 1, wherein the first plurality of device design parameters and the second plurality of device design parameters includes at least two or more of:
a number of cutting blades of the cutting instrument;
a length of each of the cutting blades of the cutting instrument;
a cutting angle of each of the cutting blades of the cutting instrument;
a relief angle of a cutting edge of each of the cutting blades of the cutting instrument;
a curvature of each of the cutting blades of the cutting instrument;
a number of notches of each of the cutting blades of the cutting instrument;
a depth of each of the notches of each of the cutting blades of the cutting instrument;
a first cross-sectional thickness of each of the cutting blades taken at or adjacent a middle of each cutting blade; and
a second cross-sectional thickness of each cutting blade taken at or adjacent a connection with a hub.

9. The method of claim 1, further comprising:
altering one or more of the second plurality of device design parameters to a third plurality of device design parameters for the cutting instrument;
performing a third analysis with the computing device using the third plurality of device design parameters, the operation data and the bone data; and
outputting by the computing device the design of the cutting instrument after performing at least the third analysis.

10. The method of claim 1, wherein outputting by the computing device the design of the cutting instrument includes transmitting a file to a fabrication machine configured to manufacture the cutting instrument.

11. The method of claim 1, wherein the bone data is based upon one or more of: a combination of cortical and cancellous bone of a glenoid of the patient, a combination of sclerotic and osteoporotic bone of the glenoid of the patient and an angle of approach of the cutting instrument to the bone.

12. The method of claim 1, wherein the operation data includes one or more of a maximum torque applied on the cutting instrument when removing the bone, a minimum torque applied on the cutting instrument when removing the bone, a ramping of torque of the cutting instrument when removing the bone, a speed of rotation of the cutting instrument when removing the bone and an oscillation rate of the cutting instrument when removing the bone.

13. The method of claim 1, wherein at least one of the first plurality of device design parameters and the second plurality of device design parameters are determined using a machine learning engine and wherein the computing device is configured to train the machine learning engine using related prior surgical procedures including at least one result or action taken by the computing device and at least one corresponding outcome.

14. A system for designing a cutting instrument for removing a portion of a bone of a patient, comprising:
at least one processor; and
at least one memory storing instructions that, when executed by the at least one processor, cause the at least one processor to perform actions comprising:
receive bone data regarding a composition of the bone;
receive operation data regarding one or more operating parameters for the cutting instrument;
receive a first plurality of device design parameters for the cutting instrument;
perform a first analysis using the first plurality of device design parameters for the cutting instrument, the bone data and the operation data;
alter one or more of the first plurality of device design parameters to a second plurality of device design parameters for the cutting instrument;
perform a second analysis using the second plurality of device design parameters, the bone data and the operation data; and
output a design of the cutting instrument after performing at least the first analysis and the second analysis.

15. The system of claim 14, wherein the instructions, when executed by the at least one processor, cause the at least one processor to produce a file used to fabricate a physical model based upon the design of the cutting instrument.

16. The system of claim 14, wherein at least one of the first analysis and the second analysis includes instructions that when executed by the at least one processor, cause the at least one processor to simulate a loading of the cutting instrument and the bone by simulating one or more forces and moments on the cutting instrument and the bone.

17. The system of claim 16, wherein simulation of the loading of the cutting instrument and the bone includes simulating a torsion load, a compression load and a shear load to the cutting instrument and the bone.

18. The system of claim 17, wherein the instructions that when executed by the at least one processor, cause the at least one processor to determine a first one or more portions of the cutting instrument and the bone that are subject to a maximum stress based upon simulation of the loading of the cutting instrument and the bone.

19. The system of claim 18, wherein the instructions that when executed by the at least one processor, cause the at least one processor to determine a deformation or strain value of a second one or more portions of the cutting instrument and the bone based upon simulation of the loading of the cutting instrument and the bone.

20. The system of claim 19, wherein the bone data is derived from at least one of an anatomic library of data and a scan of the bone of the patient, wherein the first plurality of device design parameters and the second plurality of device design parameters includes at least two or more of:
a number of cutting blades of the cutting instrument;
a length of each of the cutting blades of the cutting instrument;
a cutting angle of each of the cutting blades of the cutting instrument;
a relief angle of a cutting edge of each of the cutting blades of the cutting instrument;
a curvature of each of the cutting blades of the cutting instrument;
a number of notches of each of the cutting blades of the cutting instrument;
a depth of each of the notches of each of the cutting blades of the cutting instrument;
a first cross-sectional thickness of each of the cutting blades taken at or adjacent a middle of each cutting blade; and
a second cross-sectional thickness of each cutting blade taken at or adjacent a connection with a hub.
